(19) United States
(12) Patent Application Publication
(10) Pub. No.: US 2010/0318139 A1
(43) Pub. Date: Dec. 16, 2010

(54) METHOD AND APPARATUS FOR ARTHROSCOPIC ROTATOR CUFF REPAIR USING TRANSOSSEOUS TUNNELS

(76) Inventor: Marc Beauchamp, Montreal (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(21) Appl. No.: 12/785,153

(22) Filed: May 21, 2010

Related U.S. Application Data
(60) Provisional application No. 61/187,477, filed on Jun. 16, 2009.

(51) Int. Cl. A61B 17/56; A61B 17/04

(52) U.S. Cl. 606/86 R; 606/148

(57) ABSTRACT

There is provided a device for use in making an arthroscopic rotator cuff repair comprising an awl having a gripping portion and a shaft for transmitting force to a hooked bone-penetrating portion for forming bone tunnels, the bone-penetrating portion having a sharp tip. The awl may also function as a suture passer and comprise an eyelet situated below the tip for receiving a suture and passing it through the curved bone tunnel. A kit comprising a suture passer of substantially the same shape and of a size as the awl is also provided to pass suture through the bone tunnel formed by the awl. The suture passer is preferably blunt. Methods of using the device and kit are also provided. The method may comprise piercing the bone in a first and second location to form a bone tunnel of substantially continuous curvature and passing a suture therethrough.

### CROSS REFERENCE TO RELATED APPLICATION

This application claims the benefit of priority of U.S. Provisional Patent Application No. 61/187,477, filed Jun. 16, 2009, which is incorporated herein by reference in its entirety.

### FIELD OF THE INVENTION

Generally, there is provided a device and method for surgery. More specifically, there is provided a device and method for making an arthroscopic rotator cuff repair.

### BACKGROUND OF THE INVENTION

Rotator cuff tear is usually an avulsion of one or more tendons from the bone (humerus). It is an extremely frequent disorder that can be secondary either to a trauma or to degeneration, such as age related degeneration. When repair is indicated, it is necessary to suture back the soft tissue (tendon) to the hard tissue (bone), thereby promoting the formation of scar tissue. The time-honoured classical method (“open approach”) is to expose both structures by an open approach that usually involves some detachment (and subsequent repair) of the deltoid muscle. Strong sutures are passed through a bone tunnel in the greater tuberosity of the humerus using a curved device (needle or suture passer). The same suture is then passed through the tendon and a knot is tied to secure it to the bone.

With the advent of arthroscopy, it became possible to perform rotator cuff repair without having to perform an “open approach” and therefore avoiding most complications related to detachment of the deltoid muscle. This technique, being also less painful and better tolerated by the patient, is becoming more and more popular. However, it is considered a much more technically demanding procedure for the surgeon than the open technique, and instead of passing sutures through bone tunnels (considered too difficult to achieve with existing instruments), suture anchors, made of metal or plastic loaded with heavy suture, are used to secure the soft tissue to the bone. These suture anchors may carry specific risks (migration, foreign body reaction, osteolysis, etc), and involve the use of a disposable screwdriver for each implantation. Many still consider the transosseous bone tunnel as the gold standard for rotator cuff repair.

Techniques for arthroscopic rotator cuff repair are known in the art.

U.S. Pat. No. 5,584,839 discloses, according to the abstract, a drill guide having a cannulated guide shaft, a sliding shaft mounted thereon, and a handle for pulling back the sliding shaft. The sliding shaft comprises a hook for holding bone between the hook and the cannulated guide shaft. Pulling back on the sliding shaft holds the bone while a drill is passed through the drill guide to form a channel. The tissue repair is completed by passing suture ends through the tunnel. However the bone tunnel is relatively short and shallow, which may result in increased fragility. Further, the device must be placed onto the deltoid muscle, raising the possibility of injury.

U.S. Pat. No. 5,681,333 discloses, according to the abstract, a similar straight cannulated drill guide having a sliding aiming arm for aligning a multifunctional drillhook. The aiming arm is lockable with respect to the drill guide and has a radiused (curved) end for clearing the greater tuberosity. The drillhook marks the drillhook exit point and may have a spiked end for stabilizing the drill guide. After drilling the tunnel, a curved perforating suture hook (which terminates in 70 degrees of arc), is used to pass the suture through the rotator cuff (tendon) and into a position for the drillhook to retrieve and pull it through the tunnel. However, here too the bone tunnel is straight and relatively shallow and may be more fragile. Placement of the device during the operation may damage the deltoid muscle.

U.S. Patent Publication 20070208356 discloses, according to the abstract, a surgical apparatus for use in arthroscopic procedures comprising drill guides for drilling first and second perpendicular intersecting holes. The apparatus may also comprise a suture retriever for retrieving from the second tunnel a suture which has been fed into the first tunnel. However, this device requires the surgeon to form two holes which meet at an exact point and requires the suture to be fed around a corner of about 90 degrees, which is known to be problematic and to make retrieval more difficult.

U.S. Patent Publication 20070088362 discloses an instrument for guiding and positioning implants within the body comprising a guide channel connected with a hook. The guide channel is disposed in a body member, the longitudinal axis of which is aligned with the hook. During use, the hook may be placed on or around bone and a drill system may be used to create a passage through bone which terminates at a point near the end of the hook. The hook may be hollow for passage of a suture loop which may be grabbed and pulled through the bone tunnel by a grabber. However, here too the bone tunnel is straight and the placement of the instrument onto bone could damage muscles.

These previous approaches are also characterized by instruments with moving parts which may be expensive and which may pose sterilization problems. It would be advantageous to have a simple apparatus for making arthroscopic rotator cuff repairs which would allow strong tunnels to be formed in a shape which would facilitate the passing of sutures during surgery. It would also be advantageous for such a device to pose less of a risk for tissue damage.

### SUMMARY OF THE INVENTION

It is an object of the invention to obviate or mitigate at least one disadvantage of previous approaches.

In one aspect, there is provided a device for making arthroscopic rotator cuff repairs. In one embodiment, there is provided A device for use in making an arthroscopic rotator cuff repair comprising an awl comprising a gripping portion for manipulating said awl, and a shaft for transmitting force to a hooked bone-penetrating portion for creating a curved bone tunnel, the hooked bone-penetrating portion comprising a sharp tip for penetrating bone.

In another aspect, there is provided a kit for making rotator cuff repairs. In one embodiment, there is provided a kit comprising the device of claim 1 together with a suture passer comprising: a gripping portion for manipulating said suture passer, said suture passer having a shaft coupled to a hooked portion for passing through said bone tunnel, said hooked portion of said suture passer having a tip defining an eyelet situated towards said tip for receiving a suture, the suture passer being of substantially the same shape as said awl and of a cross-sectional thickness which is less than or equal to said awl.

In another aspect, there is provided a method of making an arthroscopic rotator cuff repair using the above apparatus. In one embodiment, there is provided A method of making an arthroscopic rotator cuff repair comprising the steps of
penetrating said greater tuberosity of a humerus with an awl to form a curved bone channel, awl comprising a gripping portion for manipulating said awl, and a shaft for transmitting force to a hooked bone-penetrating portion for creating said curved bone tunnel, the hooked bone-penetrating portion comprising a sharp tip for penetrating bone;
passing a suture through said curved bone tunnel; and
securing a tendon to said greater tuberosity with said suture.

Other aspects and embodiments will become clear to a skilled person upon reading the following description in conjunction with the Figures.

### BRIEF DESCRIPTION OF THE DRAWINGS

Embodiments of the invention will be described, by way of example, with reference to the Figures, wherein:

FIG. 1A depicts a bone-penetrating awl, according to one embodiment;

FIG. 1B depicts a bone-penetrating awl, according to another embodiment;

FIG. 1C depicts a bone-penetrating awl, according to a further embodiment;

FIG. 1D depicts a bone-penetrating awl, according to another embodiment;

FIG. 2A depicts a suture passer, according to one embodiment;

FIG. 2B depicts a suture passer, according to another embodiment;

FIG. 2C depicts a suture passer, according to a further embodiment;

FIG. 2D depicts a suture passer, according to another embodiment;

FIG. 2E depicts a tip of a suture passer depicted as E in FIGS. 2B to 2D, according to one embodiment;

FIG. 2F depicts a tip of a suture passer depicted as F in FIG. 2A, according to another embodiment;

FIG. 3A depicts a step of a surgical method, in accordance with one embodiment;

FIG. 3B depicts a step of a surgical method, in accordance with one embodiment;

FIG. 3C depicts a step of a surgical method, in accordance with one embodiment; and

FIG. 3D depicts a step of a surgical method, in accordance with one embodiment.

### DESCRIPTION OF THE INVENTION

In one embodiment, there is provided a device for use in making an arthroscopic rotator cuff repair comprising an awl having a gripping portion and a shaft for transmitting force to a hooked bone-penetrating portion for forming bone tunnels, the bone-penetrating portion having a sharp tip. The awl may also function as a suture passer and comprise an eyelet situated below the tip for receiving a suture and passing it through the curved bone tunnel. In another embodiment, a kit comprising a suture passer of substantially the same shape and of a size as the awl is also provided to pass suture through the bone tunnel formed by the awl. The suture passer is preferably blunt. Methods of using the device and kit are also provided in another embodiment. The method may comprise piercing the bone in a first and second location to form a bone tunnel of substantially continuous curvature and passing a suture therethrough.

Bone-Penetrating Awl

In one aspect, there is provided a device for making arthroscopic rotator cuff repairs.

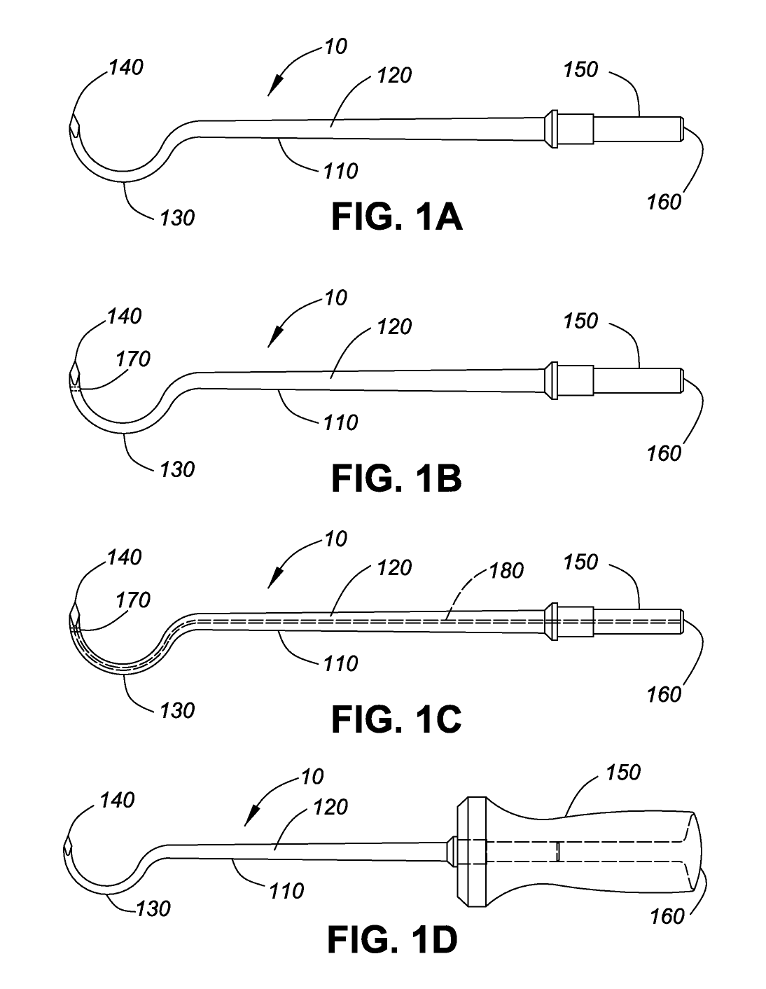

In one embodiment, the device comprises a bone-penetrating awl. Referring to FIG. 1A, the awl 10 comprises a gripping portion for manipulating the awl and shaft 110 having a substantially straight proximal portion 120, the shaft being for transmitting bone-penetrating force to a bone-penetrating hooked portion 130 for creating a bone tunnel. The bone-penetrating hooked portion has a tip 140 for penetrating bone. Preferably, the penetrating awl 10 is a handheld instrument, with the gripping portion comprising a handle 150, which may be formed thereon. Being preferably handheld, the apparatus may be inserted and manipulated manually, without the use of power tools, so force can be controlled by the surgeon at all times, which may decrease the risk of iatrogenic bone damage

The hooked bone-penetrating portion has a selected radius of curvature of 10 to 14 mm, most preferably about 12 mm. Numerical values expressed herein with the term “about” are intended to encompass the recited value plus or minus 5%. The hooked portion comprises 140 to 200 degrees, preferably 150 to 200 degrees, more preferably 160 to 200 degrees, more preferably 170 to 200 degrees, more preferably 180 to 200, or more preferably still 185 to 195 degrees of arc. Preferably, it comprises about 189 degrees of arc. In one embodiment, the hooked portion comprises 189.5 degrees of arc. It may have a curvature similar to a curved needle. The size of the hook may depend on the age and size of the patient. The curvature of the hooked portion allows for an all-arthroscopic technique, which is minimally invasive and may result in fewer complications or be less painful than an “open” approach.

The tip 140 is sharp in order to penetrate bone. The tip 140 comprises a point. Preferably, it is substantially diamond shaped. Even more preferable, the diamond shape may comprise three, four, or five sides. The tip may terminate 0 to 4 mm or, preferably, 1 to 3 mm beyond 180 degrees of arc. Most preferably, it terminates about 2 mm beyond 180 degrees of arc. The tip may be curved in conformity with the curve of the hooked portion (as in FIGS. 1B to 1D), or, preferably, it may be substantially straight (see, for example, FIG. 1A). Most preferably, as in FIG. 1A, it may be straight and project tangentially from the point at which is it joined to the curved portion of the hook.

The hooked bone-penetrating portion 130 of the shaft may taper towards the tip 140. The substantially straight portion 120 may taper towards the hooked bone-penetrating portion 130. A skilled person would appreciate that the hooked portion must have a minimum diameter to withstand the forces generated during percussion. The substantially straight portion may taper from 5.6-6.25 mm to 1.8-2.5 mm. Preferably, the substantially straight portion tapers from about 6.25 mm to about 2.5 mm.

It is preferred that the awl may have a substantially round, square, rectangular, diamond-shaped or oval in cross-sectional shape. Preferably the awl has a round cross-sectional shape in order to facilitate turning of the instrument inside the bone tunnel, for example, during formation in order to correct the direction of the tip, if required.

In one embodiment, the awl also functions as a suture passer. Referring to FIG. 1B, the hooked portion 130 of awl 10 may define an eyelet 170 extending through the width of a portion of the hooked portion, for receiving a suture. Preferably, the eyelet 170 is located just below the tip 140. The eyelet may be any shape suited to receiving 2 or 3 heavy sutures. The eyelet may be round. Preferably, the eyelet is oval. Even more preferable, the eyelet is sized about 1.25 mm×1.95 mm.

In another embodiment (see FIG. 1C), eyelet 170 is in communication with a channel 180 defined by shaft 110 and extending therethrough to an opening defined in the proximal end 160, the channel being for receiving and passing a suture. Preferably, the channel should be sized to receive two or three heavy sutures. The channel may extend through the length of the shaft 110, as shown in FIG. 1C. However, it may also extend through a shorter portion of the shaft to an opening defined elsewhere on the shaft.

In the embodiments in which the awl also functions as a suture passer, a skilled person would appreciate that the awl comprising an eyelet or channel would also need to be of sufficient hardness to permit perforation of bone, for example, by percussion.

The shaft 110 or handle 150 preferably comprises a flat surface to allow percussion, for example, with a mallet. For example, the proximal end 160 of the shaft may be flat to allow percussion. In a preferred embodiment, depicted in FIG. 1D, the handle 150 may be of greater diameter than the shaft 110 and may be shaped and sized to facilitate gripping. It may be formed of a material, such as silicone, rubber, plastic, or metal to facilitate gripping and may also be textured for the same purpose. Preferably, it is formed of silicone.

The awl may be formed of stainless steel. Preferably, it is formed of stainless steel 455. Preferably, it is in H900 condition. More preferably, it has a hardness minimum of 49 HRC (Rockwell scale). Due to the simplicity of construction, the awl may be readily sterilized, reused, and, therefore, is more “environmentally friendly”.

Suture Passer

In another aspect, there is provided a kit for making arthroscopic rotator cuff repairs comprising the above-described device together with a suture passer.

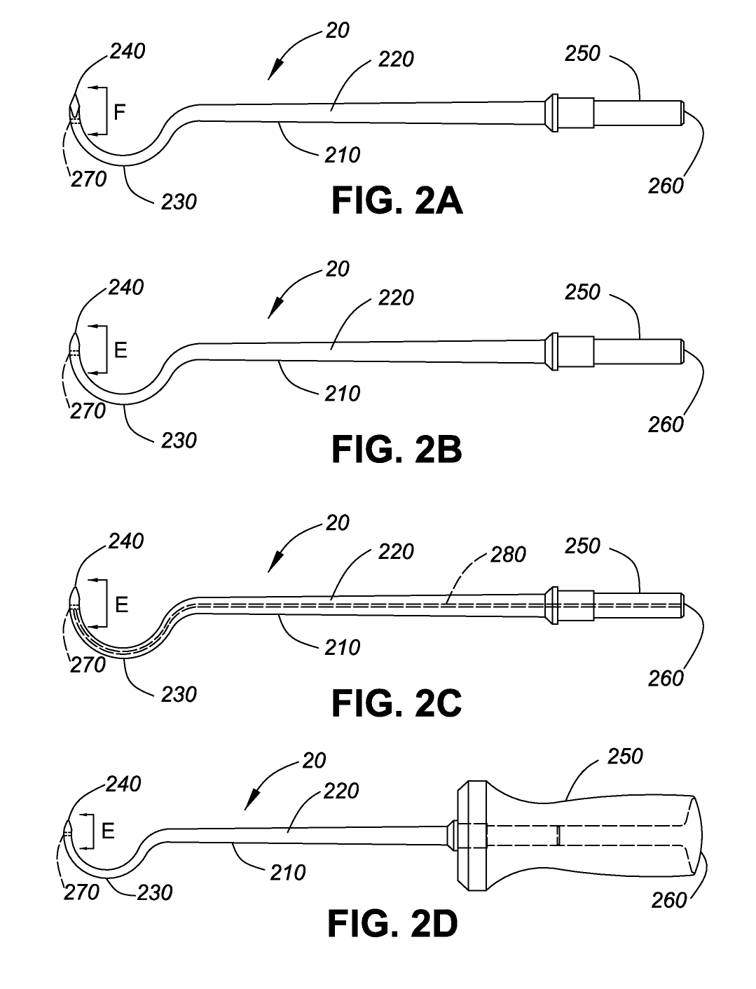

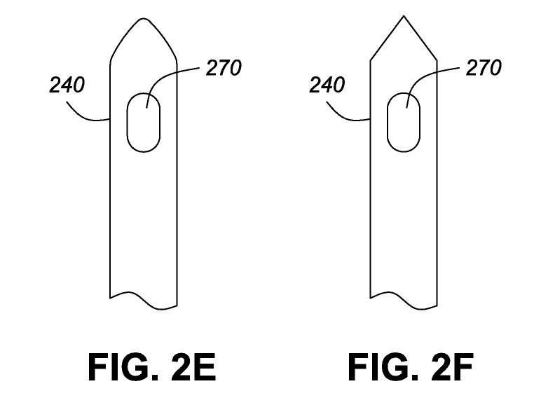

Referring to FIG. 2A, in one embodiment, the suture passer 20 is provided to pass a suture through a bone tunnel formed by the awl. The suture passer 20 comprises a gripping portion for manipulating the suture passer and a shaft 210 having a substantially straight proximal portion 220 coupled to a hooked portion 230 having a tip 240.

The suture passer is preferably of substantially the same shape and cross-sectional size as the awl described above. “Substantially the same shape”, as referred to herein, indicates that the suture passer is shaped so that its hooked portion is able to fit into (and its tip pass through) a bone channel formed by the awl described above. In some embodiments, the suture passer may also be of a smaller cross-sectional size than the awl.

An eyelet 270 is defined by the hooked portion for receiving a suture. Preferably, the eyelet 270 is situated towards the tip 240. Preferably, the eyelet is in communication with a channel extending through the width of a portion of the hooked portion so that a suture may be passed therethrough. Preferably, as depicted, for example, in FIGS. 2E and 2F, the eyelet oval, though it may also be round. Even more preferably, it is oval and sized about 1.25 mm×1.95 mm.

The hooked portion has a selected radius of curvature of 10 to 14 mm, most preferably about 12 mm. The hooked portion comprises 150 to 200 degrees, preferably 160 to 200 degrees, more preferably 170 to 200 degrees, more preferably still 180 to 200 degrees, even more preferably 190 to 200 degrees, or even more preferably still 195 to 20 degrees of arc. Preferably, it comprises about 199 degrees of arc. In one embodiment, the hooked portion comprises about 199 degrees of arc. It may have a curvature similar to a curved needle. The size of the hooked portion may depend on the age and size of the patient.

Preferably, the suture passer has a substantially round cross-sectional shape. However, it may also have a square, diamond-shaped or oval cross-sectional shape. Whatever the cross-sectional shape, a skilled person would appreciate that the suture passer is sized to fit into the curved bone tunnel formed by the awl.

As seen in FIGS. 2B and 2E, the tip 240 of suture passer 20 is preferably blunt. More preferably, it is a rounded point or round. However, it may also be pointed, as may be seen in FIG. 2A or 2F.

It is preferred that the hooked portion 230 of the suture passer be longer than the hooked bone-penetrating portion 130 of the awl to facilitate passage of the suture through the curved bone tunnel. Tip 240 may extend 0 to 6 mm beyond 180 degrees of arc. Preferably, tip 240 extends 2 to 6 mm beyond 180 degrees of arc. Even more preferable, tip 240 terminates about 4 mm beyond 180 degrees of arc.

Referring to FIG. 2C, in some embodiments, eyelet 270 may be in communication with a channel defined by the shaft 210 and extending therethrough. The channel is for receiving a suture and passing it therethrough. Preferably, it should be sized to receive two or three heavy sutures. The channel may pass through the length of the shaft 210 and terminate at an opening in the proximal end 260 (as in FIG. 2C). However, it may also pass through a shorter portion of the shaft and to an opening defined elsewhere on the shaft.

The hooked end 230 of the shaft may taper towards the tip 240. The substantially straight portion 220 may taper towards the hooked end 230. The substantially straight portion may taper from 3-6.5 mm to 1.2 to 2.5 mm. Preferably, the substantially straight portion tapers from 6.25 mm to 2.5 mm.

The shaft 210 or handle 250 comprises a flat surface to allow percussion, for example, with a mallet. Preferably, the proximal end of the shaft 260 may be flat to allow percussion. In a preferred embodiment, depicted in FIG. 2D, the handle 250 may be of greater diameter than the shaft 210 and may be shaped and sized to facilitate gripping. It may be formed of a material, such as silicone, rubber, metal, or plastic, to facilitate gripping and may also be textured for the same purpose. Preferably, it is formed of silicone.

The suture passer may be formed of stainless steel, such as stainless steel 455 in H900 condition. It may have a hardness minimum of 49 HRC (Rockwell scale). Owing to the simplicity of construction, the suture passer may be readily sterilized, reused, and, therefore, more “environmentally friendly”. However, in another embodiment, the suture passer may be disposable and formed of aluminum or plastic.

Method of Making an Arthroscopic Rotator Cuff Repair

In another aspect, there is provided a method of making an arthroscopic rotator cuff repair using the aforementioned apparatus or kit.

In some embodiments, the method comprise steps of
penetrating said greater tuberosity of a humerus with an awl to form a curved bone channel, said awl comprising a shaft having a substantially straight proximal portion and a hooked distal portion comprising a sharp tip for penetrating bone;
passing a suture through said curved bone tunnel; and
securing a tendon to said greater tuberosity with said suture.

By creating a bone tunnel, the use of foreign bodies (e.g. anchors), and the specific complications related to their use may be avoided. For example, by avoiding suture anchors, some embodiments may decrease significantly the cost of execution of the surgical procedure

In some embodiments, the step of passing the suture is performed using the above-described penetrating awl which also functions as a suture passer.

In other embodiments, the step of passing the suture is carried out with a suture passer as described above.

In certain embodiments, the penetrating step is accomplished by percussing said awl, preferably with a mallet.

In some embodiments, the penetrating step comprises:
penetrating said greater tuberosity in a first location to form a first partial curved bone tunnel, and
penetrating said greater tuberosity at second location to form a second partial curved bone tunnel in communication with said first partial bone tunnel.

When the bone is relatively hard, one or both penetrating steps may involve initial penetration of the first millimetre of bone with a straight punch or drill.

It is preferred that said first and second partial curved bone tunnels together form a curved bone tunnel having a substantially continuous curvature. Owing to the shape of the bone tunnel (which reflects the shape of the hooked portion of the awl), in some embodiments, there is provided stronger bone tunnels in fragile or osteoporotic bone than straight, shallow tunnels (which may be weaker) or suture anchors (which tend to pull out) could provide. Further, the substantially continuous curvature facilitates passage of one or more sutures, since there are no corners to negotiate.

It is preferable that said first location be a superior aspect of said greater tuberosity and said second location (the “lateral portal”) be the lateral distal cortex of the humerus. Preferably, the second location may be 1 cm or more below the proximal tip of the greater tuberosity. More preferably, the second location is about 1 cm below the proximal tip of said greater tuberosity.

The penetrating step may involve placing the tip against said greater tuberosity at an angle of about 90 degrees and penetrating the bone by manually pressing the awl into the bone. The penetration may also be awled by percussion, for example, the awl may be placed with the tip at an angle which is less than 90 degrees so that, when the awl is percussed, a component of the percussive force is directed against the bone surface to penetrate it.

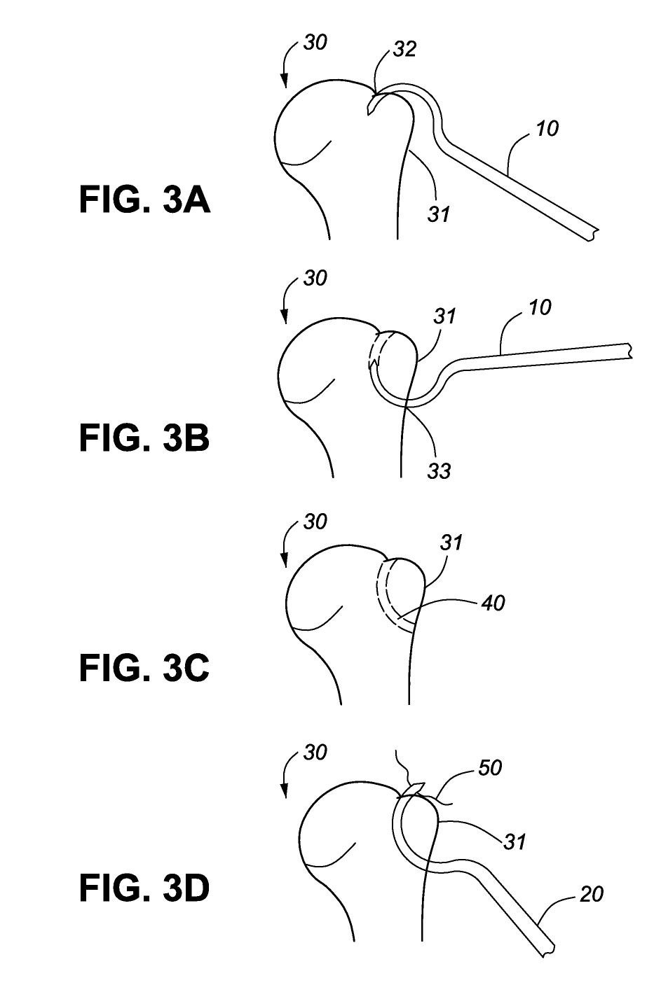

Referring to FIGS. 3A to 3D, in some embodiments, the method may comprise an arthroscopic visualisation and preparation of the humeral greater tuberosity, followed by an introduction of the awl by the lateral portal. Referring to FIG. 3A, the superior aspect 32 of the tuberosity 31 of humerus 30 may be pierced by the awl 10 at the expected exit to form a first partial bone tunnel. Then, referring to FIG. 3B, the awl 10 may be placed at the lateral distal cortex 33 of the humerus 30 and percussive perforation may be gently made to form the second partial bone tunnel. In some embodiments, the awl may be placed 1 cm below the proximal tip of the tuberosity 31 for the second perforation. The tip of the awl may penetrate the bone in the direction of the first tunnel and be visualised when the two tunnels are joined. Referring to FIG. 3C, the awl may then be retracted from the patient to yield a complete bone tunnel 40 of substantially continuous curvature. Referring to FIG. 3D, the curved suture passer 20 loaded with one or more sutures 50 (which may, in some embodiments, be heavy sutures such as #2 nonresorbable sutures or tape) may engage the tunnel, for example, from the distal entry, and may transport a portion of the suture to the proximal exit. Once the sutures 50 are visualised at the proximal exit, they may be grasped and pulled. The suture may then be passed through the tendon, and an arthroscopic knot made to bring the tendon in contact with the bone and create the desired level and area of compression. Due to the simplicity of the implements, some embodiments allow complete visualisation of the whole surgical procedure, in a strictly safe zone relatively far from the axillary nerve, thereby decreasing risks of iatrogenic nerve damage.

The above-described embodiments of the invention are intended to be examples only. Variations, alterations and modifications can be made to the particular embodiments described herein by those of skill in the art without departing from the scope of the invention, which is defined on the basis of the claims appended hereto.

What is claimed is:

1. A device for use in making an arthroscopic rotator cuff repair comprising
an awl comprising a gripping portion for manipulating said awl, and a shaft for transmitting force to a hooked bone-penetrating portion for creating a curved bone tunnel, the hooked bone-penetrating portion comprising a sharp tip for penetrating bone.

2. The device of claim 1 wherein
said hooked bone-penetrating portion of said awl comprises 140 to 200, 150 to 200, 160 to 200, 170 to 200, 180 to 200, or 185 to 195 degrees of arc.

3. The device of claim 2 wherein
said hooked bone-penetrating portion of said awl comprises about 189 degrees of arc.

4. The device of claim 3 wherein
said hooked bone-penetrating portion comprises a radius of curvature of about 12 mm.

5. The device of claim 1, wherein
said sharp tip is substantially diamond shaped.

6. The device of claim 1, wherein
said hooked bone-penetrating portion defines an eyelet for receiving a suture situated towards said tip.

7. The device of claim 6, wherein
said eyelet is oval and sized about 1.25 mm×1.95 mm.

8. The device of claim 6, wherein
said eyelet is in communication with a channel defined by said shaft and extending through at least a portion of the length of said shaft to an opening defined in the shaft, said channel for receiving and passing the suture therethrough.

9. A kit comprising the device of claim 1 together with a suture passer comprising:
a gripping portion for manipulating said suture passer, said suture passer having a shaft coupled to a hooked portion for passing through said bone tunnel, said hooked portion of said suture passer having a tip defining an eyelet situated towards said tip for receiving a suture,
the suture passer being of substantially the same shape as said awl and of a cross-sectional thickness which is less than or equal to said awl.

10. The kit of claim 9, wherein
said hooked portion of said suture pass is longer than said hooked portion of said awl.

11. The kit of claim 10 wherein
said hooked portion of said suture passer comprises 150 to 200, 160 to 200 degrees, 170 to 200 degrees, 180 to 200 degrees, 100 to 210, or 195 to 200 degrees or arc.

12. The kit of claim 10, wherein
said hooked portion of said awl comprises about 189 degrees of arc and said hooked portion of said suture passer comprises about 199 degrees of arc.

13. The kit of claim 12, wherein
said hooked portion of said awl and said hooked portion of said suture passer each have a radius of curvature of about 12 mm.

14. The kit of claim 9, wherein
said eyelet is oval and sized about 1.25 mm×1.95 mm.

15. The kit of claim 9, wherein
said eyelet is in communication with a channel defined by said shaft of said suture passer and extending through at least a portion of the length of said shaft of said suture passer to an opening defined in said shaft, said channel being for receiving and passing the suture therethrough.

16. A method of making an arthroscopic rotator cuff repair comprising the steps of
penetrating said greater tuberosity of a humerus with an awl to form a curved bone channel, awl comprising a gripping portion for manipulating said awl, and a shaft for transmitting force to a hooked bone-penetrating portion for creating said curved bone tunnel, the hooked bone-penetrating portion comprising a sharp tip for penetrating bone;
passing a suture through said curved bone tunnel; and
securing a tendon to said greater tuberosity with said suture.

17. The method of claim 16, wherein
said passing step is accomplished with said awl, said hooked portion of said awl defining an eyelet for receiving a suture therethrough.

18. The method of claim 16, wherein
said passing step is accomplished with a suture passer of substantially the same shape as said awl, said suture passer comprising a gripping portion for manipulating said suture passer, said suture passer having a shaft coupled to a hooked portion for passing through said bone tunnel, said hooked portion of said suture passer having a tip defining an eyelet situated towards said tip for receiving a suture.

19. The method of claim 16, wherein
said penetrating step comprises:
penetrating said greater tuberosity in a first location to form a first partial curved bone tunnel, and
penetrating said greater tuberosity at second location to form a second partial curved bone tunnel in communication with said first partial bone tunnel, said first and second partial curved bone tunnels together having a substantially continuous curvature.

20. The method of claim 19 wherein
one or both of said penetrating steps comprise making an initial penetration into said bone with a straight punch or drill.
